(19) United States
(12) Patent Application Publication
(10) Pub. No.: US 2011/0160830 A1
(43) Pub. Date: Jun. 30, 2011

(54) IMPLANTABLE LEADS WITH AN AXIAL REINFORCEMENT MEMBER

(76) Inventors: Kimberly A. Morris, Minneapolis, MN (US); Eduardo Carlo Lopez, Minneapolis, MN (US); Andrew De Kock, Andover, MN (US); Joel Grover, St. Paul, MN (US); Joshua Haarer, Hugo, MN (US); Timothy R. Jackson, Minneapolis, MN (US); Ronald W. Kunkel, Jim Falls, WI (US); David R. Wulfman, Minneapolis, MN (US)

(21) Appl. No.: 12/953,094

(22) Filed: Nov. 23, 2010

Related U.S. Application Data
(60) Provisional application No. 61/291,551, filed on Dec. 31, 2009.

(51) Int. Cl. A61N 1/05

(52) U.S. Cl. 607/119; 607/116

(57) ABSTRACT

Implantable electrical leads including an axial reinforcement member are disclosed. In some embodiments, an implantable electrical lead can have a body, one or more electrodes, a cable conductor, a conductor coil, and a reinforcement member. A cable conductor can be disposed within the body and is configured to convey electrical signals between the proximal region and the distal region of the lead. The reinforcement member may be coupled to or integrally formed within the lead body and is configured to limit elongation of the lead body in response to a tensile force.

### CROSS REFERENCE TO RELATED APPLICATIONS

This application claims the benefit under 35 U.S.C. §119 of U.S. Provisional Application No. 61/291,551, filed on Dec. 31, 2009, entitled “Implantable Leads with an Axial Reinforcement Member,” which is incorporated herein by reference in its entirety for all purposes.

### TECHNICAL FIELD

Various embodiments of the present invention generally relate to implantable medical devices. More specifically, embodiments of the present invention relate to implantable leads with an axial reinforcement member.

### BACKGROUND

When functioning properly, the human heart maintains its own intrinsic rhythm and is capable of pumping adequate blood throughout the body's circulatory system. However, some individuals have irregular cardiac rhythms, referred to as cardiac arrhythmias, which can result in diminished blood circulation and cardiac output. One manner of treating cardiac arrhythmias includes the use of a pulse generator (PG) such as a pacemaker, an implantable cardioverter defibrillator (ICD), or a cardiac resynchronization (CRT) device. Such devices are typically coupled to a number of conductive leads having one or more electrodes that can be used to deliver pacing therapy and/or electrical shocks to the heart. In atrioventricular (AV) pacing, for example, the leads are usually positioned in a chamber of the heart or within a blood vessel leading into or from the heart (e.g., a coronary vein), and are attached via lead terminal pins to a pacemaker or defibrillator which is implanted pectorally or in the abdomen.

### SUMMARY

Discussed herein are various implantable electrical leads including an axial reinforcement member. In Example 1, an implantable electrical lead comprises a body having a length, a proximal region with a proximal end, and a distal region with a distal end. The lead further includes a first electrode and second electrode coupled to the distal region of the lead body, a first conductor disposed within the body and configured to convey electrical signals to the first electrode, and a second conductor disposed within the body and configured to convey electrical signals to the second electrode. A reinforcement member coupled to or integrally formed within the lead body is configured to limit elongation of the lead body in response to a tensile force, wherein a distal end of the reinforcement member is positioned proximally to the second electrode.

In Example 2, the implantable electrical lead according to Example 1, wherein the body includes a tube with at least one lumen, and the reinforcement member is positioned within one of the at least one lumens.

In Example 3, the implantable electrical lead according to Example 2 or 1, wherein the reinforcement member is a monofilament.

In Example 4, the implantable electrical lead according to Example 3, wherein the monofilament comprises polytetrafluoroethylene, ethylene tetrafuoroethylene, or silicone.

In Example 5, the implantable electrical lead according to Example 2 or 1, wherein the reinforcement member is a multifilament braid.

In Example 6, the implantable electrical lead according to Example 5, wherein the multifilament braid comprises polyethylene terephthalate.

In Example 7, the implantable electrical lead according to Example 1, wherein the reinforcement member is a polymeric tube integrally formed within a wall of the body.

In Example 8, the implantable electrical lead according to Example 7, wherein the reinforcement member comprises high modulus silicone or polyethylene terephthalate.

In Example 9, the implantable electrical lead according to any of Examples 1-8, wherein the reinforcement member comprises a non-conductive material, and wherein the distal end of the reinforcement member is connected to the second electrode and the proximal end of the reinforcement member is connected to insulation within the body.

In Example 10, an implantable medical device comprises a lead body having a length, a proximal region with a proximal end, and a distal region with a distal end, the proximal end including a terminal connector configured to attach to an implantable device. The implantable medical device further includes a plurality of electrodes coupled to the distal region of the lead body, and a plurality of conductors disposed within the lead body and configured to convey electrical signals between the proximal region and the distal region of the lead body. A reinforcement member coupled to or integrally formed within the lead body is configured to limit elongation of the lead body in response to a tensile force, and wherein the reinforcement member crosses at least one distal electrode within the distal region of the lead body.

In Example 11, the implantable medical device according to Example 10, wherein the proximal end of the lead body is made from polyurethane and the distal end of the lead body is made from silicone.

In Example 12, the implantable medical device according to Example 10 or 11, wherein the reinforcement member extends lengthwise along the silicone portion of the lead body.

In Example 13, the implantable medical device according to Example 10 or 11, wherein the lead body includes a tube located longitudinally along at least a portion of the length of the lead body, wherein the reinforcement member is located within the tube.

In Example 14, the implantable medical device according to any of Examples 10-13, wherein the reinforcement member is a monofilament or multifilament braid.

In Example 15, the implantable medical device according to any of Examples 10-14, further including a pacemaker or a cardiac defibrillator.

In Example 16, an implantable electrical lead configured to convey electrical signals between a heart and a pulse generator comprises a body having a length, a polyurethane proximal region and a silicone distal region. The lead further includes a plurality of electrodes coupled to the distal region of the lead body, and a cable conductor disposed within the proximal region of the lead body and configured to convey electrical signals between the proximal region and the distal region. A non-conductive reinforcement member coupled to or integrally formed within insulation located within the lead body is configured to limit elongation along at least a portion of the length of the lead body in response to a tensile force. A distal end of the reinforcement member can be attached to the insulation proximally to the first electrode. A proximal end of the reinforcement member can be attached to the insulation proximal to or distal to a distal most electrode.

In Example 17, the implantable electrical lead according to Example 16, wherein the non-conductive reinforcement member is a monofilament comprising polytetrafluoroethylene, ethylene tetrafuoroethylene, or high modulus silicone.

In Example 18, the implantable electrical lead according to Example 16, wherein the non-conductive reinforcement member is a multifilament braid made from polyethylene terephthalate.

In Example 19, the implantable electrical lead according to Example 16, wherein the non-conductive reinforcement member is a polymeric tube integrally formed along at least a portion of the distal region of the body.

In Example 20, the implantable electrical lead according to Example 19, wherein the polymeric tube has a diameter between about 20/1000 inch and about 30/1000 of an inch.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic view of a cardiac rhythm management system in accordance with an embodiment of the present invention;

FIG. 2 is a schematic view illustrating an exemplary lead in accordance with one or more embodiments of the present invention;

FIGS. 3A and 3B are longitudinal cross-sectional views of a portion of an exemplary lead in accordance with one or more embodiments of the present invention illustrating the elongation of silicone relative to the proximal electrode in response to an axial load;

FIGS. 4A and 4B are longitudinal cross-sectional views of a distal portion of a lead with a reinforcement member in accordance with various embodiments of the present invention;

FIGS. 5A and 5B are longitudinal cross-sectional views of a distal region of a lead and corresponding cutaway views of the distal region in accordance with some embodiments of the present invention; and

FIGS. 6A-6E are end, cross-sectional views showing several illustrative implantable leads including a reinforcement member.

The drawings have not necessarily been drawn to scale. For example, the dimensions of some of the elements in the figures may be expanded or reduced to help improve the understanding of the embodiments of the present invention. While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

In a typical lead embodiment, the distal electrode is both mechanically and electrically attached to a coil conductor. Often a second coil is connected to a proximal electrode. In some implementations, silicone insulation within the lead body not only isolates the distal conductor from the proximal conductor, but also bridges the proximal electrode to provide increased axial strength and limit the overall elongation of the coil. In these cases, the conductors and insulation are all able to translate under load and return back to where they started upon removal of the load.

However, new leads are constantly being developed that have a reduced lead body diameter and/or additional electrodes. One method for reducing lead body diameter is to replace some or all of the coil conductors with stranded wire conductors such as cables. However, stranded wire conductors in combination with coil conductors impart new challenges. For example, coils are able to elongate and translate during the application of an axial load. As a result, the relative spring constant of a cable is much higher than that of a coil and thus the cable does not stretch under loading. Though the cable and the electrode it is attached to do not move, the coil and silicone are still free to elongate. As a result, under loading the coil and the silicone translate relative to a stationary cable and the electrode. When the load is removed, the components may not restore to their original positions.

As explained in further detail below, various embodiments of the present invention relate to an implantable electrical lead including a reinforcement member that provides additional load support to the lead. In the following description, for the purposes of explanation, numerous specific details are set forth in order to provide a thorough understanding of embodiments of the present invention. It will be apparent, however, to one skilled in the art that embodiments of the present invention may be practiced without some of these specific details.

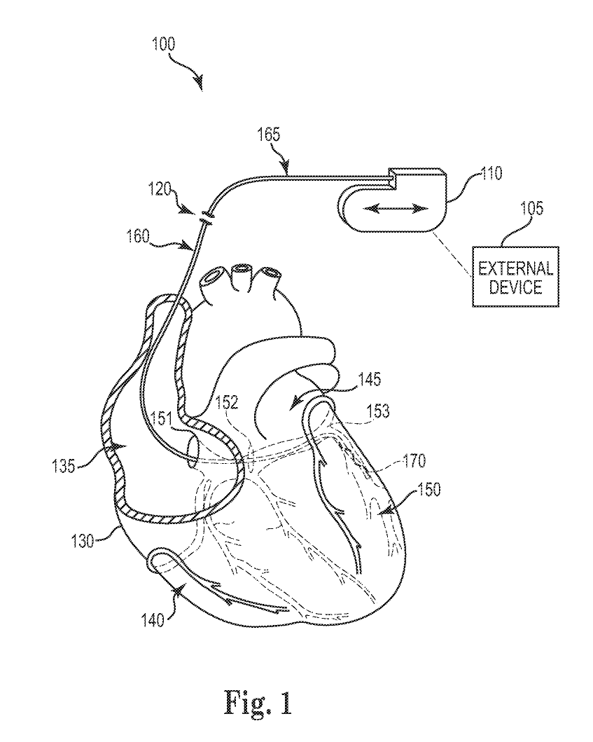

FIG. 1 is a schematic view of a cardiac rhythm management (CRM) system 100 in accordance with an embodiment of the present invention. In the embodiment shown in FIG. 1, the CRM system 100 includes one or more external devices 105, a pulse generator (PG) 110 and an implantable lead 120 coupled to the PG 110. The PG 110 can communicate with the one or more external device(s) 105. The PG 110 can be a device such as a pacemaker, ICD, cardiac resynchronization therapy device with defibrillation capabilities (a CRT-D device), or a comparable device. In some embodiments, the PG 110 includes both pacing and defibrillation capabilities. The PG 110 can be implanted within the body, typically at a location such as in the patient's chest or abdomen.

The external device(s) 105 may be a local or remote terminal or other device (e.g., a computing device and/or programming device), operable to communicate with the PG 110 from a location outside of the patient's body. According to various embodiments, external device 105 can be any device external to the patient's body that is telemetry enabled and capable of communicating with the PG 110. Examples of external devices can include, but are not limited to, programmers (PRM), in-home monitoring devices, personal computers with telemetry devices, MRI scanner with a telemetry device, manufacturing test equipment, or wands. In some embodiments, the PG 110 communicates with the remote terminal 105 via a wireless communication interface. Examples of wireless communication interfaces can include, but are not limited to, radio frequency (RF), inductive, and acoustic telemetry interfaces.

The lead 120 has a lead body 160 that includes a proximal region 165 and a distal region 170. The lead 120 can be implanted in the patient's heart 130, which as shown in FIG. 1, includes a right atrium 135, a right ventricle 140, a left atrium 145, and a left ventricle 150. In the embodiment illustrated in FIG. 1, the distal end 170 of the lead 120 is transvenously guided through the right atrium 135, through the coronary sinus ostium 151, and into a branch of the coronary sinus 152 or great cardiac vein 153. The illustrated position of the lead 120 can be used for sensing or for delivering pacing and/or defibrillation energy to the left side of heart 130, or to treat arrhythmias or other cardiac disorders requiring therapy delivered to the left side of the heart 130. Additionally, the lead 120 can also be used to provide treatment in other regions of the heart 130 (e.g., the right ventricle 140 or right atrium 135).

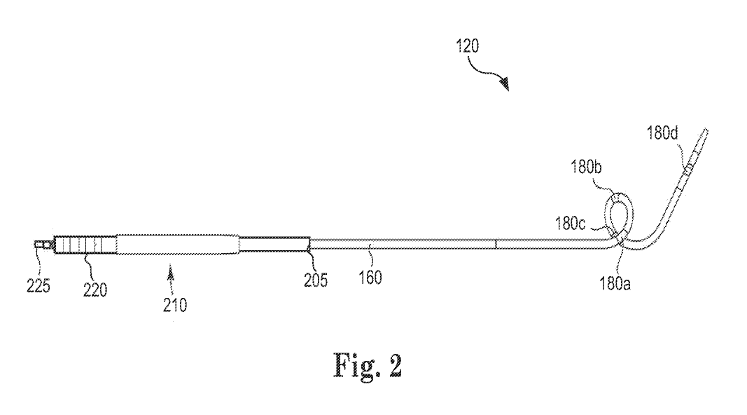

FIG. 2 is a schematic view showing the exemplary lead 120 of FIG. 1 in greater detail. As further shown in FIG. 2, the lead 120 includes a terminal connector assembly 210, which is coupled to a proximal end 205 of the lead body 160 and to the pace/sense electrodes 180a-180d.

In the illustrated embodiment, the connector assembly 210 includes a connector body 220 and a terminal pin 225. The connector assembly 210 is coupled to the lead body 160 and can be configured to mechanically and electrically couple the lead to a header on PG 110 (see FIG. 1). In various embodiments, the terminal pin 225 extends proximally from the connector body 220, and in some embodiments is coupled to an inner conductor coil that extends longitudinally through the lead body 160 to one or more pace/sense electrodes or ring electrodes 180a-180d. In some embodiments, the pace/sense electrode(s) can include a tip electrode (not shown) located at the distal-most extremity of the lead 120. In other embodiments, the lead 120 may include additional pace/sense electrodes located more proximally along the lead 120.

The pace/sense electrodes 180a-180d can be made of any suitable electrically conductive material such as ELGILOY, MP35N, tungsten, tantalum, iridium, platinum, titanium, palladium, stainless steel, as well as alloys of any of these materials.

In some embodiments, the distal electrode 180d is both mechanically and electrically attached to an inner conductor coil. In some embodiments, a second coil is connected to a proximal and adjacent electrode 180c. Silicone insulation not only isolates the distal electrode 180d from the proximal electrode 180c, but it also bridges the proximal electrode 180c to provide increased axial strength and limit the overall elongation of the coil. In this case, the conductors and insulation are all able to translate under load and return back to where they started upon removal of the load.

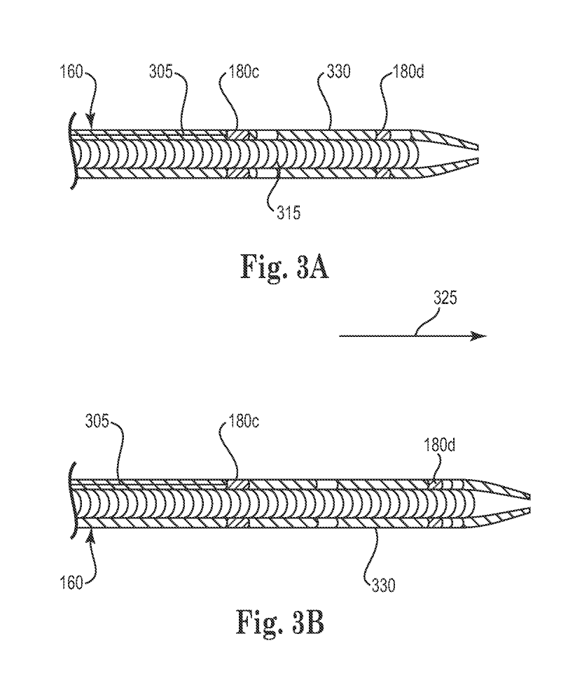

FIGS. 3A-3B are schematic views showing the elongation of the lead body 160 relative to the proximal electrode 180c in response to an axial load asserted on the lead. FIGS. 3A and 3B represent, for example, the lead body 160 prior to and subsequent to being subjected to an axial load, respectively. Examples of axial loads include forces experienced during implantation and resulting from patient movements. As shown in a first view in FIG. 3A, the lead body 160 includes a cable 305 attached to a first electrode 180c. A conductor coil 315 connects the first electrode 180c with the second electrode 180d. When an axial load 325 is applied, and as further shown in FIG. 3B, the coil 315 and the silicone 330 translate relative to cable 305 and the first electrode 180c. When the load is removed, the components may not restore to their original positions.

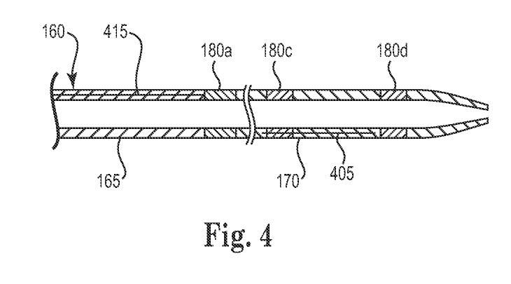

FIG. 4 shows the distal portion of lead body 160 with a reinforcement member 405 that may be used in accordance with various embodiments of the present invention. In some embodiments, reinforcement member 405, sometimes referred to as an axial support or tether, may be a made from silicone. In use, the reinforcement member 405 can run lengthwise within the lead body 160, and is configured to withstand tensile forces that typically occur after implantation of the lead body 160 within the patient's body. The reinforcement member 405 can also be configured to withstand the tensile forces on the lead body 160 that can occur after implantation without impacting any conductors, joints, or functional electrical insulation.

In accordance with some embodiments, the proximal portion 165 of the flexible lead body 160 can be made from polyurethane while the distal portion 170 can be made from silicone. As describe above in FIG. 1, the distal portion 170 can include one or more electrodes such as 180a-180d. As illustrated in FIG. 4, a polyurethane/silicone 410 transition can occur when the material of the flexible lead body 160 changes. In some embodiments, for example, a transition in materials may be desirable to increase the flexibility in the distal section of the lead body 160.

In some embodiments, as shown in FIG. 4, a cable conductor 415 can be disposed within the proximal region 170 of the lead body 160, and is configured to convey electrical signals between the proximal region and the distal region of the lead. The distal end of cable conductor 415 can terminate by attaching to the first electrode 180a. The electrodes are not traditionally electrically coupled to one another, though in some versions they could be. The distal most electrode 180d can be electrically coupled by the conductor coil to the terminal pin (not shown.)

In accordance with various embodiments, the reinforcement member 405 can be non-conductive. The reinforcement member 405 can be coupled to, or integrally formed within, insulation located within the flexible lead body 160. The reinforcement member 405 is typically made from a higher modulus material than the silicone used for the insulation and therefore is configured to limit elongation along at least a portion of the length of the lead body 160 in response to a tensile force. In some embodiments, as illustrated in FIG. 4, a proximal end of the reinforcement member 405 is attached to the insulation proximally to the electrode adjacent (in this case, electrode 180c) to the distal most electrode 180d. A distal end of the reinforcement member 405, in turn, is attached to the insulation proximal to the distal most electrode 180d.

Some embodiments use a reinforcement member 405 that is continuously attached to the lead body insulation. In one or more embodiments, the reinforcement member 405 can span the cable 415 termination at a “proximal” non-moving electrode to prevent translation of the silicone under the electrode. According to various embodiments, reinforcement member 405 can be compliant enough to stretch with the lead body insulation (while still resisting elongation) and/or the adhesion between the stiffening member and lead body insulation is strong enough such that the bond does not break under application of load.

In some additional embodiments, the reinforcement member 405 can be rigidly connected to one or more of the electrode(s). In other embodiments, the reinforcement member 405 can be a cable that mechanically (but not electrically) connects to the distal electrode 180d and to some other feature of the lead 120. In some embodiments, the reinforcement member 405 is a non-conductive material that is connected to the distal most electrode 180d and to another feature of the lead 12 proximal to electrode 180d. In various embodiments, the reinforcement member 405 is discretely connected at both ends, and in some embodiments is connected to the lead body insulation. For example, in some embodiments, a reinforcement member (conductive or non-conductive) can be mechanically, but not electrically, connected to the distal electrode 180d and connected to the polyurethane of the flexible lead body 160 through the use of a mechanical joint.

In accordance with various embodiments, a higher modulus reinforcement member 405 can be integrated into the distal lead body to limit elongation of the lead body between electrodes. For example, the stiffer reinforcement member 405 can have a durometer greater than the remainder of the lead body insulation, which is typically 50-70 durometer silicone. The flexibility of the reinforcement member 405 can depend on a variety of factors such as, for example, the size of the reinforcement member 405, specific lead configurations, and the like. Consider, for example, a reinforcement member 405 in a tubing form. This particular reinforcement member will take up more space in the lead cross-section and therefore, the modulus would have to be lower than if the reinforcement member 405 was a filament.

In some cases, elongation of the insulation and other material under and around the reinforced proximal electrode 180c can be minimized and/or prevented while not impacting, or minimally impacting, other functions of the lead (e.g. size, bending stiffness). The reinforcement member 405 can be made from a variety of materials. In some embodiments, the reinforcement member 405 can be a polymeric monofilament made from polytetrafluoroethylene, ethylene tetrafluoroethylene, or high modulus silicone. In other embodiments, the reinforcement member 405 can be a polymeric multifilament braid or weave made from polyethylene terephthalate and/or other materials. In some embodiments, the reinforcement member 405 can be a polymeric tube such as a high modulus silicone tube or a polyethylene terephthalate/silicone tube. Still yet, in other embodiments, the reinforcement member 405 can be a wire or stranded wire (e.g. a cable).

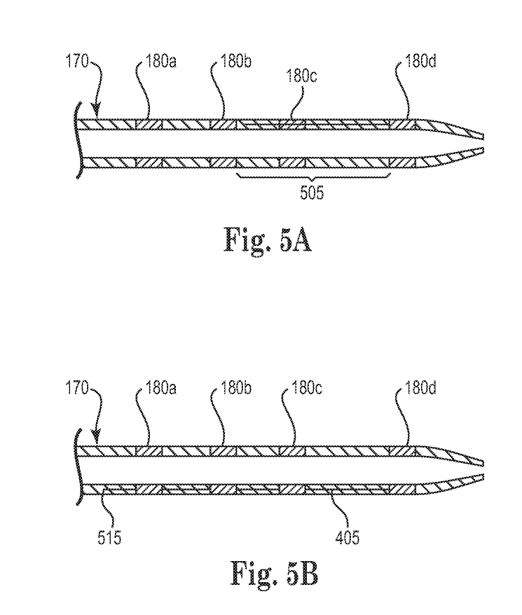

FIGS. 5A and 5B show the distal region 170 of lead 120 and corresponding cutaway views of the distal region 170 in accordance with some embodiments of the present invention. According to various embodiments, reinforcement member 405 spans across section 505 which includes the next to last distal most electrode 180c connected to a cable conductor. In the embodiment illustrated in FIG. 5A, the reinforcement member 405 is connected proximal side of the distal most electrode (180d) and to some feature of the lead 120 or some feature (e.g., a proximal electrode, insulation, or other feature) proximal to electrode 180c. In some cases, the proximal and distal ends of the reinforcement member 405 can be extended to a proximally adjacent electrode 180a or distally within the lead body assembly 510. FIG. 5B shows an alternative embodiment in which the reinforcement member 405 extends past several proximal electrodes 180a-180d, and the proximal end of the reinforcement member attaches at attachment point 515 on the proximal side of electrode 180a (or, in some embodiments, proximal to electrode 180a).

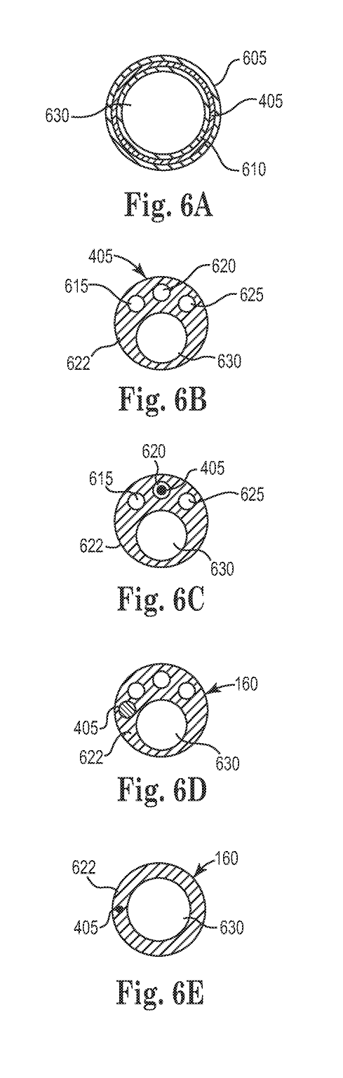

FIGS. 6A-6E is cross-sectional views showing several illustrative implantable leads including a reinforcement member 405. FIG. 6A shows reinforcement member 405 as a tube integrally formed within flexible lead body 160. In some embodiments, reinforcement member 405 can be a braid. According to one or more embodiments, the reinforcement member 405 can be embedded within the flexible lead body 160 through a process such as overmolding or co-extrusion. As such, the outer silicone 605 can be joined to the inner silicone layer 610 through the middle reinforcement member layer 405 in some embodiments. The middle reinforcement member layer 405, in various embodiments, can be a polymeric material with a pattern of holes or cutouts for the silicone to bridge the middle layer.

The resulting tube formed by the overmolding process has a lumen 630 configured to receive a coil conductor. The lumen 630 can be designed to provide a compression fit or a slight clearance for the coil conductor. In some embodiments, the diameter of the tube can range from approximately 20/1000 of an inch to approximately 30/1000 of an inch. In at least one embodiment, the diameter of the tube is approximately 26/1000 of an inch.

FIG. 6B shows a cross-section of lead 120 with a multi-lumen design in which the reinforcement member 405 comprises an internal tubular member 622. For example, the inner tubular member can be made of a high modulus silicone. Each of the internal lumens 615, 620, 625, and 630 allow for the use of multiple conductors for supplying current to the electrodes. For illustrative purposes, the inner tubular member 622 is shown with three lumens 615, 620 and 625 having the same diameter and one lumen 630 having a different diameter allowing for placement of various conductors and/or reinforcement members. In other embodiments, however, the relative dimensions and/or locations of the lumens 615, 620, 625, and 630 may vary from that shown. In addition, the inner tubular member 622 may include a greater or lesser number of lumens, depending on the particular configuration of the lead 120. For example, the inner tubular member 620 may include a greater number of lumens to house additional conductors for supplying current to other electrodes.

FIG. 6C shows reinforcement member 405 positioned within one of the lumens 615, 620 or 625 instead of, or in addition to, the inner tubular member 622 being made of the high modulus silicone. In some embodiments, the reinforcement member 405 can transverse a portion of, or the entire length of, the lumen 615, 620 or 625. In some embodiments, the reinforcement member 405 may be attached to the insulation and/or flexible lead body at discrete points or continuously using a medical adhesive.

FIG. 6D-6E shows the reinforcement member 405 disposed within a wall f the flexible lead body 160 (e.g., via over molding or co-extrusion). In some embodiments, the reinforcement member 405 can be a polymeric monofilament or braid made from polytetrafluoroethylene, ethylene tetrafluoroethylene, or high modulus silicone. In other embodiments, the reinforcement member 405 can be a polymeric multifilament braid or weave made from polyethylene terephthalate and/or other materials.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. An implantable electrical lead, comprising:
a lead body having a length, a proximal region with a proximal end, and a distal region with a distal end;
a first conductor extending within the lead body from the proximal end in a direction towards the distal end of the lead body;
a second conductor extending within the lead body from the proximal end in a direction towards the distal end of the lead body;
at least a first electrode and a second electrode coupled to the distal region of the lead body, the first electrode operatively coupled to the first conductor and the second electrode operatively coupled to the second conductor; and
a reinforcement member coupled to or integrally formed within the lead body and configured to limit elongation of the lead body in response to a tensile force, wherein a distal end of the reinforcement member is positioned proximally to the second electrode.

2. The implantable electrical lead of claim 1, wherein the body includes a tube with at least one lumen, and the reinforcement member is positioned within one of the at least one lumens.

3. The implantable electrical lead of claim 1, wherein the reinforcement member is a monofilament.

4. The implantable electrical lead of claim 2, wherein the monofilament comprises polytetrafluoroethylene, ethylene tetrafuoroethylene, or silicone.

5. The implantable electrical lead of claim 1, wherein the reinforcement member is a multifilament braid.

6. The implantable electrical lead of claim 5, wherein the multifilament braid comprises polyethylene terephthalate.

7. The implantable electrical lead of claim 1, wherein the reinforcement member is a polymeric tube integrally formed within walls of the body.

8. The implantable electrical lead of claim 7, wherein the reinforcement member comprises high modulus silicone or polyethylene terephthalate.

9. The implantable electrical lead of claim 1, wherein the reinforcement member comprises a non-conductive material, and wherein the distal end of the reinforcement member is connected to the second electrode and the proximal end of the reinforcement member is connected to insulation within the body.

10. An implantable medical device, comprising:
a lead body having a length, a proximal region with a proximal end, and a distal region with a distal end, the proximal end including a terminal connector configured to attach to an implantable device;
a plurality of conductors extending within the lead body;
a plurality of electrodes located on the distal region of the lead body, each electrode operatively coupled to one of the plurality of conductors extending within the lead body; and
a reinforcement member coupled to or integrally formed within the lead body and configured to limit elongation of the lead body in response to a tensile force, and wherein the reinforcement member crosses at least one distal electrode within the distal region of the lead body.

11. The implantable medical device of claim 10, wherein the proximal end of the lead body is made from polyurethane and the distal end of the lead body is made from silicone.

12. The implantable medical device of claim 11, wherein the reinforcement member extends lengthwise along the silicone portion of the lead body.

13. The implantable medical device of claim 10, wherein the lead body includes a tube located longitudinally along at least a portion of the length of the lead body, wherein the reinforcement member is located within the tube.

14. The implantable medical device of claim 10, wherein the reinforcement member is a monofilament or multifilament braid.

15. The implantable medical device of claim 10, further including a pacemaker or a cardiac defibrillator coupled to the lead.

16. An implantable electrical lead to convey electrical signals between a heart and a pulse generator, wherein the implantable electrical lead comprises:
a lead body having a length, a polyurethane proximal region and a silicone distal region;
a plurality of electrodes coupled to the silicone distal region of the lead body;
a cable conductor disposed within the polyurethane proximal region of the body and configured to convey electrical signals between the polyurethane proximal region and the silicone distal region; and
a non-conductive reinforcement member coupled to or integrally formed within insulation located within the body and configured to limit elongation along at least a portion of the length of the lead body in response to a tensile force, and wherein a distal end of the reinforcement member is attached to the insulation proximal to the first electrode and a proximal end of the reinforcement member is attached to the insulation proximal to or distal to a distal most electrode.

17. The implantable electrical lead of claim 16, wherein the non-conductive reinforcement member is a monofilament comprising polytetrafluoroethylene, ethylene tetrafuoroethylene, or high modulus silicone.

18. The implantable electrical lead of claim 16, wherein the non-conductive reinforcement member is a multifilament braid made from polyethylene terephthalate.

19. The implantable electrical lead of claim 16, wherein the non-conductive reinforcement member is a polymeric tube integrally formed along at least a portion of the silicone distal region of the body.

20. The implantable electrical lead of claim 19, wherein the polymeric tube has a diameter between about 20/1000 inch and about 30/1000 of an inch.
